(19) United States
(12) Patent Application Publication
(10) Pub. No.: US 2010/0129831 A1
(43) Pub. Date: May 27, 2010

(54) METHODS FOR DIAGNOSING HYPERSENSITIVITY REACTIONS

(76) Inventor: Sarah Brenner, Tel Aviv (IL)

Correspondence Address: FENNEMORE CRAIG, 3003 NORTH CENTRAL AVENUE, SUITE 2600, PHOENIX, AZ, 85012, US

(21) Appl. No.: 12/632,651

(22) Filed: Dec. 7, 2009

Related U.S. Application Data
(63) Continuation of application No. PCT/IL2008/000771, filed on Jun. 5, 2008.
(60) Provisional application No. 60/990,298, filed on Nov. 27, 2007.
(60) Provisional application No. 60/990,296, filed on Nov. 27, 2007.
(60) Provisional application No. 60/990,295, filed on Nov. 27, 2007.
(60) Provisional application No. 60/944,541, filed on Jun. 18, 2007.
(60) Provisional application No. 60/942,539, filed on Jun. 7, 2007.

(51) Int. Cl. G01N 33/53

(52) U.S. Cl. 435/7.1

(57) ABSTRACT

The invention is directed to the diagnosis of allergic reactions. Particularly, the invention is directed to in vitro assays for diagnosing systemic hypersensitivity reactions, and identifying agents causing these reactions.

### RELATED APPLICATION DATA

This application is the U.S. continuation of PCT/IL2008/000771, filed Jun. 5, 2008, which claims the benefit of U.S. Provisional Application Nos. 60/990,298, filed Nov. 27, 2007; 60/990,296, filed November 27, 2007; 60/990,295 filed Nov. 27, 2007; 60/944,541 filed Jun. 18, 2007; and 60/942,539 filed Jun. 7, 2007, the contents of each of which are herein incorporated by reference for all purposes.

### FIELD OF THE INVENTION

The invention is directed to the diagnosis of allergic reactions. Particularly, the invention is directed to in vitro assays for diagnosing and identifying causative agents of systemic hypersensitivity reactions.

### BACKGROUND OF THE INVENTION

Interferon (IFN) gamma proteins are well known in the art. Interferon gamma variants are known and can be measured collectively or separately (Maher S G et al. Interferon: cellular executioner or white knight? Curr Med Chem. 2007; 14(12):1279-89).

Allergic diseases are disorders associated with exaggerated immune response to a foreign antigen. In the case of allergies to inhaled antigens, common symptoms associated with such disorders include rhinorrhea, sneezing, and nasal congestion (upper respiratory tract); wheezing and dyspnea (lower respiratory tract); and itching (eyes, skin). Signs may include nasal turbinate edema, sinus pain on palpation, wheezing, conjunctival hyperemia and edema, and skin lichenification. Stridor, wheezing, and sometimes hypotension are life-threatening signs of anaphylaxis that may result from severe allergies.

Allergic disorders are currently considered a substantial public health concern, due to their high incidence (20 to 30% of the population) and lack of effective remedies. Thus, considerable efforts have been devoted for developing reliable assays for diagnosis of allergic disorders, identification of the allergen(s) associated with these pathologies, detection and monitoring of ongoing allergic responses, and assessment of predisposition for developing such disorders.

Food Hypersensitivity

Food hypersensitivity, or food allergy, is an adverse immunologic reaction to dietary proteins or other antigens. Most adverse reactions to food are mediated by other, nonimmune, mechanisms, which should be distinguished from food allergy, e.g. lactose intolerance due to lactase deficiency, irritable bowel syndrome, and infectious gastroenteritis. True food allergy, by contrast can be mediated by humoral (e.g. IgE antibodies), cellular (e.g. T-cells and basophils), or both components of the immune system. In addition to typical foods, food hypersensitivity has been described as associated with herbal and homeopathic drugs.

Immune-mediated adverse reactions to food antigens can be divided into distinct clinico-pathologic entities based on presentation (immediate or delayed), target organ specificity, and pathogenic mechanisms. The reactions are manifested by a range of different clinical signs, depending on the allergen, mechanism, and patient age. Generally, IgE-mediated allergy develops during infancy, is acute in onset, while T cell-mediated reactions typically manifest gradually and are chronic.

Interstitial Nephritis

Tubulointerstitial nephritis is a primary injury to renal tubules and interstitium, resulting in decreased renal function. The disease can be caused by a variety of triggers such as genetic diseases, metabolic obstructive uropathy, and exposure to chronic environmental toxins, drugs, and herbs. Cell-mediated immune reactions may play a role in the disease mechanism (Spanou et al., 2006). Treatment and prognosis vary by the etiology and potential for reversibility of the disorder at the time of diagnosis. The disease can be caused by allergic drug reactions, identification of which would greatly aid in treatment of this disease.

Acute interstitial nephritis (AIN) is characterized by a sudden impairment of renal function, mild proteinuria, and sterile pyuria. It is commonly caused by drugs such as antibiotics (lactams, sulfonamides, aminoglycosides, and quinolones), anticonvulsants, diuretics (thiazide and furosemide), proton pump inhibitors (omeprazole), nonsteroidal anti-inflammatory drugs (Markowitz et al., 2005), and alternative medications (Colson et al., 2005); this form is known as “drug-induced interstitial nephritis (DIN).” The art does not teach reliable in vitro assays for accurate screening methods for involvement of allergic reactions in AIN.

Implant-Associated Hypersensitivity

Medical implants, devices and prostheses are used for various purposes, e.g. for providing structural/mechanical aid or body part replacement. Implants often include metal wires, rods, plates, screws, tubes, etc, which are typically comprised of stainless steel (e.g. ASTM F138, containing Fe, Ni, Cr and Mo), cobalt alloys (e.g. ASTM F75, containing Co, Ni, Cr and Mo), and titanium alloys (e.g. ASTM F136, containing Ti, Al and V). Cardiovascular implants or stents are used with balloon angioplasty to treat arterial stenosis and are typically comprised of 316L stainless steel (e.g. the Cordis Palmaz-Schatz and Crossflex™ stents), gold (e.g. gold-plated hybrid stents), cobalt chromium alloys (e.g. ASTM F1058, containing Fe, Co, Ni, Cr and Mo), tantalum (e.g. the Tantalum Cordis stent) and titanium alloys (e.g. Nitinol, containing Ni and Ti) (Hallab et al., 2001; Lim, 2004).

Allergic responses have been associated with implantation of metal components of such devices (Hallab et al., 2001). It has been suggested that degradation products of metallic biomaterials (e.g. particulate wear debris, colloidal organometallic complexes, free metallic ions, inorganic metal salts or oxides, and precipitated organometallic storage forms) activate the immune system by forming complexes with native proteins.

Metals known to be sensitizers (haptenic moieties in antigens) are beryllium, nickel, cobalt, and chromium; in addition, responses to tantalum, titanium, and vanadium have occasionally been reported. The prevalence of metal sensitivity among the general population is approximately 10% to 15%, with nickel sensitivity having the highest prevalence (approximately 14%).

Drug-eluting stents, e.g. containing paclitaxel and sirolimus (rapamycin) have recently been introduced to address restenosis. In some cases, drug-eluting stents are bare metal stents coated on the surface with a drug and a biostable polymer. Additional polymer—and non-polymer drug delivery systems are in development. Hypersensitivity adverse reactions have been reported with drug-eluting stents, resulting in dermatitis, vasculitis, urticaria, immune suppression, implant failure, and occasionally potentially fatal effects such as in-stent restenosis and late stent thrombosis. Prediction, evaluation, and monitoring of hypersensitivity responses to implanted devices would be highly valuable prior to implantation and, in case of an adverse outcome, in determining whether the device should be removed.

Allergy Testing Methods

The most commonly used methods for allergy testing in general are in vivo skin tests such as patch tests, percutaneous (prick) testing, and intradermal testing, wherein antigen is directly introduced into the skin. The prick test can detect most allergies. The intradermal test is more sensitive but less specific; it can be used to evaluate sensitivity to allergens with negative or equivocal prick test results. Besides food antigens in the case of suspected food allergies, antigens typically used are, in the case of allergic rhinitis, pollens (tree, grass, and weed), molds, house dust mites, animal danders and sera, insect venom, foods, and β-lactam antibiotics. Preferably, the subject should not have taken antihistamines before testing for a period of 3 to 20 days, depending on the type of antihistamine. Skin testing is generally contra-indicated in patients with dermographism, generalized skin lesions, or severe reactions to allergens by skin contact or inhalation. Skin tests detect the presence of IgE antibodies that bind the allergen on the cutaneous mast cells. Therefore, the utility of this test is believed to be primarily for allergies with cutaneous (skin) as opposed to gastrointestinal or respiratory pathologies.

While the negative predictive value of skin tests is relatively high (estimated to be over 95% for IgE-mediated reactions), their positive predictive value for IgE-mediated reactions is only around 50%. Hence, a positive skin test in isolation is insufficient for a diagnosis of hypersensitivity. Further, several test sessions at the practitioner's office are typically required for screening of multiple allergens, reducing patient enthusiasm and compliance.

Radioallergosorbent testing (RAST) detects the presence of allergen-specific serum IgE. A known allergen in the form of an insoluble polymer-allergen conjugate is mixed with the serum to be tested and with ¹²⁵I-labeled anti-IgE antibody. Allergen-specific IgE in the serum binds the conjugate and can be quantified by measuring ¹²⁵I-labeled antibody. This test is more expensive than skin testing and is considered less specific.

Provocative testing involves direct exposure of the mucosae to allergen and is indicated for patients who must document their reaction (e.g., for occupational or disability claims) and sometimes for diagnosis of food allergy. Nasal and bronchial challenge are primarily research tools, but bronchial challenge is sometimes used when the clinical significance of a positive skin test is unclear or when no antigen extracts are available (e.g., for occupation-related asthma).

Other potential screening methods of uncertain diagnostic value have been reported, including examination of allergen-specific immunoglobulin G (IgG), antigen-antibody complexes, leukocyte cytotoxic tests, and lymphocyte proliferation and migration tests.

T lymphocytes are known to be involved in the pathology of cutaneous adverse drug reactions (CADR), undesirable changes in the skin resulting from systemic or topical drug administration, and other pathologies associated with hypersensitivity reactions. It has been suggested that the responding T cell phenotype and in vitro and in vivo cytokine pattern might correlate with the type of immune response evolving after antigen contact. Tests wherein drug-treated T cells are assayed for secretion of macrophage migration inhibition factor (MIF) and/or interferon gamma (IFN-γ) have been evaluated in the diagnosis of CADR induced by certain drugs (Koga et al., 1995; Koga et al., 2000; Rasanen et al., 1999; Kubota et al., 1999; Livni et al., 1999; Halevy et al., 1998; Halevy et al., 1999; Halevy et al., 2000; Halevy et al., 2001; Halevy et al., 2002; Halevy et al., 2004; Goldberg et al., 2004; Wohl et al., 2004; Halevy et al., 2005; Goldberg et al., 2005; Wohl et al., 2006). These references are not relevant to the present invention, which is directed to the diagnosis and measurement of systemic immune responses.

U.S. Pat. Appl. Pub. No. 2005/0074822 is directed to a method of detecting an antigen-specific T lymphocyte in a sample, comprising combining a biological sample comprising a cell with an antigen, an IL-15 receptor agonist, and an IL-7 receptor agonist, forming a test sample, and detecting an antigen-specific T lymphocyte in the test sample. This publication suggests detection of T lymphocyte by measuring cytokine secretion, but provides no disclosure or suggestion of use of IFN-γ release for accurate screening and clinical diagnosis of allergic reactions.

A cytokine secretion assay was also disclosed for the diagnosis of a skin contact allergy, particularly to metals such as nickel (U.S. Pat. Appl. Pub. No. 2004/0115744). This reference neither discloses nor in any way suggests use of T-cell interferon-gamma release as a means of detecting systemic immune responses (i.e. internal responses not manifested cutaneously).

Lymphocyte proliferation tests (or lymphocyte transformation tests, LTT) measure antigen-induced in vitro proliferation of peripheral blood lymphocytes; but few data exist regarding their efficacy. In vitro leukocyte migration inhibition testing involves the measurement of mixed-population leukocyte migration activity; this test is only used in combination with other indicia (Hallab et al., 2001)

A number of publications disclose diagnostic methods for food hypersensitivity diagnosis. U.S. Pat. No. 6,884,625 is directed to diagnosing food allergies utilizing detection of IgE or IgA antibodies in stool samples; U.S. Pat. No. 6,858,398 is directed to diagnosing food allergies by detection of IgA antibodies in saliva, and U.S. Pat. No. 5,983,899 relates to analyzing a sample from the large intestine of a suspected allergy patient, after provoking the mucous membrane of the large intestine with an allergen.

U.S. Pat. Appl. Pub. No. 20040072272 relates to a method of diagnosing immunologic food sensitivities based on the presence of certain other disorders or immunologic diseases, the presence of certain HLA alleles, or a failure to respond to treatment for microscopic colitis.

The EuroPrevall Project was launched to improve the predictive value of food allergy tests based on allergen extracts or purified allergen molecules, testing affinity of IgE-allergen interactions, and evaluating the potential of tests such as histamine release tests or basophil activation tests performed with permanently growing cell lines. Despite such efforts, to date no in vitro test is currently believed to reliably diagnose clinical food allergy (Asero et al., 2007).

In addition, there is to date no accepted screening test for hypersensitivity to drug-eluting stent or other surgical implants. While published studies have employed scratch and patch tests for investigating the relationship between metal (contact) allergy and implant-associated adverse effects, there is concern about the applicability of skin testing for studying implant hypersensitivity, since dermal antigen exposure occurs differently from periprosthetic exposure. In addition, the test may induce hypersensitivity in a previously insensitive subject. Finally, information is lacking regarding appropriate metal challenge agents for in vivo use. Neither does the art teach or suggest using an IFN-γ release assay for assessing the compatibility of medical implants, stents, staples, or other implanted surgical devices.

Thomas et al (2006) reported use of a proliferation assay to test for titanium sensitivity in a single patient. This reference neither discloses nor in any way suggests use of T-cell interferon-gamma release as a means of detecting metal sensitivity.

In summary, currently available tests are not considered to be capable of predicting or evaluating implant-associated allergy, especially in the case of relatively subtle or atypical responses, e.g. titanium- and/or vanadium-containing implants.

Neither does the art teach or suggest using an IFN-γ release assay for assessing the safety of herbal and homeopathic drugs and additives or for diagnosing and quantifying allergic reactions involving these preparations. In general, there is scant literature on the role of herbal supplements and remedies induction or exacerbation of immune sensitivities. Thus, reliable methods for specifically diagnosing and analyzing allergic reactions to herbal preparations, medicaments and dietary supplements would be highly beneficial.

There remains a long-felt need for providing accurate, reliable, simple, safe and quantitative methods for diagnosing, predicting and quantifying allergic responses. In vitro testing methods available to date are labor-intensive and clinically unpopular. Use of IFN-γ release for accurate screening and clinical diagnosis of allergic reactions has not been disclosed. In particular, a correlation between IFN-γ release by PBL and in vivo allergic reactions has not been shown for immune disorders other than cutaneous allergic conditions such as cutaneous adverse drug reactions.

### SUMMARY OF THE INVENTION

The invention is directed to the diagnosis of allergic reactions. Particularly, the invention is directed to in vitro assays for diagnosing systemic hypersensitivity reactions, and identifying agents causing these reactions.

According to various aspects, the methods and kits of the invention are useful for detecting an allergic reaction to an antigen in a subject, for determining the identity of an allergen associated with a hypersensitivity reaction in a subject, for detecting antigen-specific T cells in a subject, and/or for quantifying the immune response of a subject to a putative allergen. In some embodiments, the methods and kits of the invention provide automated and/or high throughput analysis.

In one embodiment, the present invention provides a method for detecting an allergy or allergic reaction in a subject, the method comprising the steps of (a) incubating a T cell-containing cell population from the subject or an aliquot of the cell population in a culture media comprising a test substance, under conditions enabling or conducive to release of IFN-γ by the cell population; (b) measuring the amount of IFN-γ protein present in the culture media; and c) comparing the amount of IFN-γ protein released to a reference standard, whereby a significant increase in the amount of IFN-γ protein relative to the reference standard indicates that the subject is allergic to the test substance. In some embodiments, the T cell-containing cell population utilized is a blood-derived cell population. In another embodiment, the allergy or allergic response detected is manifested by a symptom selected from the group consisting of renal, respiratory, cardiovascular and gastrointestinal symptoms. In another embodiment, the IFN-γ protein release is quantified as antigen-specific release of IFN-γ protein. In another embodiment, an immunoassay such as ELISA is utilized. Each possibility represents a separate embodiment of the present invention.

In another embodiment, a method of the present invention further comprises the steps of (d) incubating a second T cell-containing cell population from the subject in a second culture media, wherein the second culture media comprises the test substance described above, and wherein the second cell population has been obtained from the subject at a time point subsequent to performing the above method; (e) measuring the amount of IFN-γ present in the second culture media; and (f) comparing the amount of IFN-γ protein produced by the first and second cell populations, whereby a statistically significant difference in the amount of IFN-γ protein between the two cell populations is indicative of alteration in the magnitude of the allergy, between the first and second time points.

In another embodiment of the above method, a significant increase in the amount of IFN-γ protein indicates progression or exacerbation of the allergy, while a significant decrease indicates amelioration of the allergy. In another embodiment, the amounts of IFN-γ protein produced by the two T cell-containing cell populations are each compared to an internal standard, e.g. a no-antigen control for that cell population. The difference between the two values is considered the antigen-specific release of IFN-γ protein, and this value is compared to the equivalent value for the other time point. Each possibility represents a separate embodiment of the present invention.

In another embodiment, the present invention provides a method for monitoring the severity of an allergic response, comprising the steps of measuring antigen-specific release of IFN-γ protein by T-cell containing cell populations obtained from a subject at multiple time points, as described above. As provided herein, methods of the present invention enable sufficiently accurate and quantitative assessment of allergic responses to determine the effects of environmental change. It will be understood to those skilled in the art that the present invention embraces testing of subjects at multiple time points (e.g. three, four, or five or more time points) in monitoring progression of an allergic response. Each possibility represents a separate embodiment of the present invention.

In another embodiment, the present invention provides a method for monitoring a response of an allergy to an immunotherapy, comprising the steps of measuring antigen-specific release of IFN-γ protein by T-cell containing cell populations obtained from a subject at multiple time points, as described above.

In another embodiment, the present invention provides a method for determining a predisposition of a subject to developing an allergy or allergic reaction to a test substance, the method comprising the steps of (a) incubating a T cell-containing cell population from the subject or an aliquot of the cell population in a culture media comprising the test substance, under conditions enabling or conducive to release of IFN-γ by the cell population; (b) measuring the amount of IFN-γ protein in the culture media; and (c) comparing the amount of IFN-γ protein to a reference standard, whereby a significant increase in the amount of IFN-γ protein relative to the reference standard indicates that the subject is allergic to the test substance. In another embodiment, the T cell-containing cell population utilized is a blood-derived cell population. In another embodiment, the allergy or allergic response is manifested by a symptom selected from the group consisting of renal, respiratory, cardiovascular and gastrointestinal symptoms. In another embodiment, the IFN-γ protein release is quantified as antigen-specific release of IFN-γ protein. Each possibility represents a separate embodiment of the present invention.

In other embodiments, the methods of the invention enable measurement of the immune response of a subject to an allergen in a quantitative fashion. In another embodiment, the superior quantitativeness of methods of the present invention provides a means for monitoring the progress of the subject's hypersensitivity reaction.

In another embodiment, the present invention provides a method for diagnosing hypersensitivity to a naturopathic or homeopathic medicament, comprising the step of measuring IFN-gamma release or secretion from a T cell-containing sample of the subject in the presence of the naturopathic or homeopathic medicament or a fraction or component thereof, as described herein.

In another embodiment, the present invention provides a method for quantification of an allergic response of a subject to a ingested, inhaled, or injected allergen, comprising the step of measuring antigen-specific IFN-gamma production by a T-cell containing cell population by a method of the present invention, whereby the level of antigen-specific IFN-gamma production indicates in a quantitative fashion the severity of the allergic response.

In another embodiment, the present invention provides a method for monitoring an allergy or allergic reaction to a surgical implant or stent, comprising the steps of measuring antigen-specific release of IFN-γ protein by T-cell containing cell populations obtained from a subject at multiple time points, as described herein.

In another embodiment, the present invention provides a method for monitoring a response of an allergy or allergic reaction to removal of a surgical implant or stent, comprising the steps of measuring antigen-specific release of IFN-γ protein by T-cell containing cell populations obtained from a subject at multiple time points, as described herein.

In another embodiment, the present invention provides a method for evaluating the suitability of an implant or surgical device for a subject prior to its implantation, comprising the step of measuring antigen-specific release of IFN-γ protein by a T-cell containing cell population obtained from a subject in the presence of the implant or surgical device or a component thereof, as described herein.

In another aspect, there is provided a kit suitable for carrying out a method of the invention. In one embodiment, the kit comprises (i) a plurality of test samples, each test sample containing a putative allergen; and (ii) means for detecting the presence of secreted IFN-γ, as detailed herein.

In some embodiments, the kit comprises (i) a plurality of vessels each vessel comprising a test sample containing a putative allergen as detailed herein, wherein the vessels are suitable for incubating a cell population, under conditions enabling release of IFN-γ by cells sensitized to an allergen; and (ii) means for detecting the presence of IFN-γ in each vessel. In another embodiment, the vessels are wells. In another embodiment, the vessels are test tubes. In another embodiment, the vessels are any other type of vessels suitable for methods of the present invention. Each possibility represents a separate embodiment of the present invention.

In another embodiment, the present invention provides a kit or apparatus for high-throughput allergy testing, the kit comprising:

a. a means of incubating a T cell-containing cell population from a subject in a culture media comprising a test substance, under conditions enabling release of IFN-γ by the T cell-containing cell population; and

b. a means of measuring the amount of IFN-γ protein in the culture media. In another embodiment, the kit further comprises an instruction to compare the amount of IFN-γ present to a reference standard. Each possibility represents a separate embodiment of the present invention.

In another embodiment, methods of the present invention exclude use of radioactivity, thus being amenable to inexpensive, high-volume use not requiring special facilities. Each possibility represents a separate embodiment of the present invention.

In another embodiment, the present invention provides use of:

a. a means of incubating a T cell-containing cell population from a subject in a culture media comprising a test substance, under conditions enabling release of IFN-γ by the T cell-containing cell population; and

b. a means of measuring the amount of IFN-γ protein in the culture media, in the preparation of a kit for detecting an allergy or allergic disorder in a subject, wherein the allergy or allergic disorder is manifested by a symptom selected from the group consisting of renal, respiratory, cardiovascular and gastrointestinal symptoms, whereby a significant increase in the amount of IFN-γ protein relative to the reference standard indicates that the subject is allergic to the test substance. In another embodiment, the kit further comprises an instruction to compare the amount of IFN-γ present to a reference standard. Each possibility represents a separate embodiment of the present invention.

Other objects, features and advantages of the present invention will become clear from the following description.

### DETAILED DESCRIPTION OF THE INVENTION

The invention is directed to the diagnosis of allergic reactions. Particularly, the invention is directed to in vitro assays for diagnosing systemic hypersensitivity reactions, and identifying agents causing these reactions.

According to various aspects, the methods and kits of the invention are useful for detecting an allergic reaction to an antigen in a subject, for determining the identity of an allergen associated with a hypersensitivity reaction in a subject, for detecting antigen-specific T cells in a subject, and/or for quantifying the immune response of a subject to a putative allergen. In some embodiments, the methods and kits of the invention provide automated and/or high throughput analysis.

In one embodiment, the present invention provides a method for detecting an allergy or allergic reaction in a subject, the method comprising the steps of (a) incubating a T cell-containing cell population from the subject in a culture media comprising a test substance, under conditions enabling or conducive to release of IFN-γ by the cell population; (b) measuring the amount of IFN-γ protein in the culture media; and c) comparing the amount of IFN-γ protein released to a reference standard, whereby a significant increase in the amount of IFN-γ protein relative to the reference standard indicates that the subject is allergic to the test substance. In another embodiment, a multiplicity of aliquots of the cell population are tested to ensure reproducibility. In another embodiment, additional aliquots are used for a control sample, e.g. a non-antigen control, or are stored to use a standards for subsequent testing. In another embodiment, the T cell-containing cell population utilized is a blood-derived cell population. In another embodiment, the allergy or allergic response is manifested by a symptom selected from the group consisting of renal, respiratory, cardiovascular and gastrointestinal symptoms. Preferably, the IFN-γ protein release is quantified as antigen-specific release of IFN-γ protein. Each possibility represents a separate embodiment of the present invention.

The test substance of methods and compositions of the present invention is, in another embodiment, a substance suspected of being associated with/triggering the allergy or allergic reaction in the subject, as described in more detail herein.

In another embodiment, a method of the present invention further comprises the steps of (d) incubating a second T cell-containing cell population from the subject in a second culture media, wherein the second culture media comprises the test substance described above, and wherein the second cell population has been obtained from the subject at a time point subsequent to performing the above method; (e) measuring the amount of IFN-γ protein in the second culture media; and (f) comparing the amount of IFN-γ protein produced by the first and second cell populations, whereby a statistically significant difference in the amount of IFN-γ protein between the two cell populations is indicative of alteration in the magnitude of the allergy, between the first and second time points. In another embodiment, an aliquot of a previously obtained sample is tested in parallel to use as a standard. Each possibility represents a separate embodiment of the present invention.

Typically, the conditions of the two incubations are substantially similar. For example, the second T cell-containing cell population will ordinarily be the same type of cells (e.g. PBMC) as the first T cell-containing cell population. The second culture media will ordinarily be substantially identical in composition to the first culture media and may conveniently be e.g. a second aliquot of the first culture media. Preferably, the second culture media comprises the test substance in substantially the same amount as the first culture media, and the incubation conditions for the first and second cell populations are similar or identical. Conveniently, the incubations can be performed in parallel, e.g. if the cells have been stored prior to performing the assay in a manner that maintains their viability and responsiveness. Each possibility represents a separate embodiment of the present invention.

In another embodiment of the above method, a significant increase in the amount of IFN-γ protein indicates progression or exacerbation of the allergy, while a significant decrease indicates amelioration of the allergy. In another embodiment, the amounts of IFN-γ protein produced by the two T cell-containing cell populations are each compared to an internal standard, e.g. a no-antigen control for that cell population. The difference between the two values is considered the antigen-specific release of IFN-γ protein, and this value is compared to the equivalent value for the other time point. Each possibility represents a separate embodiment of the present invention.

In another embodiment, an alteration of at least 30% in the amount of IFN-γ protein relative to the prior sample is considered a positive result. In another embodiment, an alteration of at least 25% is considered a significant alteration. In another embodiment, an alteration of at least 35% is considered a significant alteration. In another embodiment, an alteration of at least 40% is considered a significant alteration. In another embodiment, an alteration of at least 50% is considered a significant alteration. In another embodiment, an alteration of at least 60% is considered a significant alteration. In another embodiment, an alteration of at least 80% is considered a significant alteration. In another embodiment, an alteration of at least 100% (two-fold) is considered a significant alteration. In another embodiment, an alteration of at least three-fold is considered a significant alteration. In another embodiment, an alteration of at least two standard deviations (std) is considered a significant alteration. In another embodiment, an alteration of at least 2.5 std is considered a significant alteration. In another embodiment, an alteration of at least 3 std is considered a significant alteration. In another embodiment, the total amount of IFN-γ protein is compared to the prior sample. In another embodiment, the antigen-specific amount of IFN-γ protein release (calculated as described herein) is compared to that of the prior sample. Each possibility represents a separate embodiment of the present invention.

In another embodiment, the present invention provides a method for monitoring the severity of an allergic response, comprising the steps of measuring antigen-specific release of IFN-γ protein by T-cell containing cell populations obtained from a subject at multiple time points, as described above. As provided herein, methods of the present invention enable sufficiently accurate and quantitative assessment of allergic responses to determine the effects of environmental change. It will be understood to those skilled in the art that the present invention embraces testing of subjects at multiple time points (e.g. three, four, or five or more time points) in monitoring progression of an allergic response. Each possibility represents a separate embodiment of the present invention.

In another embodiment, the present invention provides a method for monitoring a response of an allergy to an immunotherapy, comprising the steps of measuring antigen-specific release of IFN-γ protein by T-cell containing cell populations obtained from a subject at multiple time points, as described above.

In another embodiment, the present invention provides a method for determining a predisposition of a subject to developing an allergy or allergic reaction to a test substance, the method comprising the steps of (a) incubating a T cell-containing cell population from the subject in a culture media comprising the test substance, under conditions enabling or conducive to release of IFN-γ by the cell population; (b) measuring the amount of IFN-γ protein in the culture media; and (c) comparing the amount of IFN-γ protein to a reference standard, whereby a significant increase in the amount of IFN-γ protein relative to the reference standard indicates that the subject is allergic to the test substance. In another embodiment, a multiplicity of aliquots of the cell population are tested to ensure reproducibility. In another embodiment, additional aliquots are used for a control sample, e.g. a non-antigen control, or are stored to use a standards for subsequent testing. In another embodiment, the T cell-containing cell population utilized is a blood-derived cell population. In another embodiment, the allergy or allergic response is manifested by a symptom selected from the group consisting of renal, respiratory, cardiovascular and gastrointestinal symptoms. In another embodiment, the IFN-γ protein release is quantified as antigen-specific release of IFN-γ protein. Each possibility represents a separate embodiment of the present invention.

In another embodiment, a method of the present invention is performed in a plurality of wells or compartments, each compartment comprising a test sample containing a putative allergen. As provided herein, methods of the present invention are amenable, in some embodiments, for use as automated and/or high throughput analyses. In another embodiment, the methods are amenable for screening dozens of samples. In another embodiment, the methods are amenable for screening hundreds of samples. In another embodiment, the methods are amenable for screening thousands of samples. Each possibility represents a separate embodiment of the present invention.

The reference standard of methods of the present invention is obtained, in another embodiment, by incubating under the same conditions an additional aliquot of the T cell-containing cell population, e.g. in an additional aliquot of the culture media, that lacks the test substance but otherwise is identical to the aliquot of the culture media used in testing the experimental sample. Such a reference standard may be referred to as a “negative control sample.” In another embodiment, the reference standard is obtained by incubating a T cell-containing cell population of a control subject (e.g. a subject not allergic to the test substance) with culture media containing the test substance. In another embodiment, the reference standard is an average response obtained from cell samples of a population (e.g. a population of subjects not allergic to the test substance or a general population of subjects). In another embodiment, the amounts of IFN-γ protein produced by the cell populations of the experimental subject and the control subject are each compared to an internal standard, e.g. a no-antigen control for that cell population. The difference between the two values is considered the antigen-specific release of IFN-γ protein, and these values are compared between the experimental subject and the control subject. Each possibility represents a separate embodiment of the present invention.

In another embodiment, an increase of at least 30% in the amount of IFN-γ protein over the reference standard is considered a positive result. In another embodiment, an increase of at least 25% is considered positive. In another embodiment, an increase of at least 35% is considered positive. In another embodiment, an increase of at least 40% is considered positive. In another embodiment, an increase of at least 50% is considered positive. In another embodiment, an increase of at least 60% is considered positive. In another embodiment, an increase of at least 80% is considered positive. In another embodiment, an increase of at least 100% (two-fold) is considered positive. In another embodiment, an increase of at least three-fold is considered positive. In another embodiment, an increase of at least two std is considered positive. In another embodiment, an increase of at least 2.5 std is considered positive. In another embodiment, an increase of at least 3 std is considered positive. In another embodiment, the total amount of IFN-γ protein is compared to the reference standard. In another embodiment, the antigen-specific amount of IFN-γ protein release (calculated as described herein) is compared to the reference standard. Each possibility represents a separate embodiment of the present invention.

Allergies and Allergic Disorders

According to some embodiments, the methods and kits of the invention are useful for evaluating hypersensitivity in a subject in need thereof. A “subject” in connection with the invention is a mammalian, preferably a human subject. In various embodiments, the subject may be an infant, a child or an adult human patient manifesting symptoms or signs associated with a hypersensitivity reaction. In another embodiment, the subject is a pediatric subject (e.g. under the age of 18). In another embodiment, the subject is a adolescent subject (e.g. 12-18). In another embodiment, the subject is a child (e.g. under the age of 10). In another embodiment, the subject is a young child (e.g. under the age of 5). In another embodiment, the subject is a baby or infant subject (e.g. under the age of 2 or under the age of 1). Each possibility represents a separate embodiment of the present invention.

In another embodiment, the allergic response diagnosed by a method of the present invention is a systemic allergic response. In another embodiment, the allergic response is manifested by an internal symptom. In another embodiment, the internal symptom is a respiratory symptom. In another embodiment, the internal symptom is a renal symptom. In another embodiment, the internal symptom is a cardiovascular symptom. In another embodiment, the internal symptom is a gastrointestinal symptom. In another embodiment, the term refers to a symptom selected from the group consisting of renal, respiratory, cardiovascular and gastrointestinal symptoms. In another embodiment, any other type of non-cutaneous symptom known in the art may be classified as an internal symptom. In another embodiment, the allergic response is not manifested by a clinical (i.e. readily detectable) cutaneous symptom (e.g. CADR). In another embodiment, the allergic response is one that does not present to the physician with any cutaneous symptoms, e.g. as a contact allergy or cutaneous adverse reaction, e.g. a drug reaction. As provided herein, the present invention has shown that allergic responses, even when not accompanied by clinically detectable cutaneous manifestations, are amenable to quantitative detection and characterization via measurement of interferon-gamma secretion by T-cells of the subject. Each possibility represents a separate embodiment of the present invention.

In another embodiment, the allergic reaction detected or monitored by methods and compositions of the present invention is manifested by a renal symptom or manifestation. In another embodiment, the allergic reaction is manifested by a respiratory symptom or manifestation. In another embodiment, the allergic reaction is manifested by a cardiovascular symptom or manifestation. In another embodiment, the allergic reaction is manifested by a gastrointestinal symptom or manifestation. In another embodiment, the allergic reaction is manifested by a gastrointestinal symptom selected from the group consisting of renal, respiratory, cardiovascular and gastrointestinal symptoms.

In another embodiment, the subject is afflicted with an allergic renal reaction, e.g. interstitial nephritis. In another embodiment, the subject is afflicted with acute interstitial nephritis. Acute tubulointerstitial nephritis is associated with an inflammatory infiltrate and edema involving the renal interstitium that often develops over days to months. Over 95% of cases result from infection or an allergic drug reaction; a syndrome of acute tubulointerstitial associated with uveitis (renal-ocular syndrome) also occurs and is idiopathic. Acute tubulointerstitial causes acute renal insufficiency or failure; severe cases, delayed therapy, or continuance of an offending drug can lead to permanent injury with chronic renal failure. In another embodiment, the interstitial nephritis is of unknown etiology. In another embodiment, the interstitial nephritis is suspected to be allergy-mediated. In another embodiment, the renal symptom of the present invention is selected from the group consisting of renal failure, flank pain, intense thirst, polyuria, oliguria, and anuria. In another embodiment, the renal symptom is any other type of renal symptom of possible allergic etiology known in the art. Each possibility represents a separate embodiment of the present invention.

In another embodiment, the present invention provides a method of determining the cause of a renal symptom, comprising the step of measuring antigen-specific interferon-gamma production by a T-cell containing cell population by a method of the present invention, as described herein, whereby a significant amount of antigen-specific IFN-gamma production indicates an allergic etiology.

In another embodiment, the present invention provides a method of determining the cause of a case of interstitial nephritis, comprising the step of measuring antigen-specific interferon-gamma production by a T-cell containing cell population by a method of the present invention, as described herein, whereby a significant amount of antigen-specific IFN-gamma production indicates an allergic etiology.

In another embodiment, the subject is afflicted with an allergic respiratory reaction, e.g. anaphylaxis or interstitial lung disease. In another embodiment, the respiratory symptom is selected from the group consisting of interstitial fibrosis, bronchiolitis obliterans organizing pneumonia, asthma, noncardiogenic pulmonary edema, pleural effusions, pulmonary eosinophilia, pulmonary hemorrhage, veno-occlusive disease, coughing, wheezing, difficulty in breathing, bronchospasm, and angioedema. In another embodiment, the respiratory symptom of the present invention is any other type of respiratory symptom of possible allergic etiology known in the art. Each possibility represents a separate embodiment of the present invention.

In another embodiment, the present invention provides a method of determining the cause of a respiratory symptom, comprising the step of measuring antigen-specific interferon-gamma production by a T-cell containing cell population by a method of the present invention, as described herein, whereby a significant amount of antigen-specific IFN-gamma production indicates an allergic etiology.

In another embodiment, the present invention provides a method of determining the cause of a case of interstitial lung disease, comprising the step of measuring antigen-specific interferon-gamma production by a T-cell containing cell population by a method of the present invention, as described herein, whereby a significant amount of antigen-specific IFN-gamma production indicates an allergic etiology.

In another embodiment, the subject is afflicted with an allergic cardiovascular reaction, e.g. arterial hypotension. In another embodiment, the cardiovascular symptom is selected from the group consisting of exertional dyspnea, fatigue, fainting, low blood pressure, dizziness, hypotension, tachycardia, cardiovascular collapse, bradycardia and cardiac arrest. In another embodiment, the cardiovascular symptom of the present invention is any other type of cardiovascular symptom of possible allergic etiology known in the art. Each possibility represents a separate embodiment of the present invention.

In another embodiment, the present invention provides a method of determining the cause of a cardiovascular symptom, comprising the step of measuring antigen-specific IFN-gamma production by a T-cell containing cell population by a method of the present invention, as described herein, whereby a significant amount of antigen-specific IFN-gamma production indicates an allergic etiology.

In another embodiment, the subject is afflicted with a gastrointestinal reaction, e.g. a food hypersensitivity reaction. In another embodiment, the gastrointestinal symptom is selected from the group consisting of nausea (e.g. postprandial nausea), vomiting, abdominal pain, cramping, diarrhea, abdominal pain, and a sensation of early satiety. In another embodiment, the gastrointestinal symptom is any other gastrointestinal symptom known in the art. Each possibility represents a separate embodiment of the present invention.

In another embodiment, the present invention provides a method of determining the cause of a gastrointestinal symptom, comprising the step of measuring antigen-specific interferon-gamma production by a T-cell containing cell population by a method of the present invention, as described herein, whereby a significant amount of antigen-specific IFN-gamma production indicates an allergic etiology.

In another embodiment, the allergic reaction of the present invention is selected from the group consisting of anaphylaxis, asthma, eosinophilic gastroenteropathy, dietary protein gastroenteropathy, and celiac disease. In another embodiment, the allergic reaction is an IgE-mediated reaction. In another embodiment, the allergic reaction is any other systemic allergic reaction known in the art. Each possibility represents a separate embodiment of the present invention.

In another embodiment, the allergic reaction of the present invention is pollen-food allergy syndrome, which is typically caused by cross-reactivity between certain pollen and food allergens.

In another embodiment, the allergic reaction of the present invention is dietary protein enterocolitis. In another embodiment, the dietary protein enterocolitis presents as weight loss or failure to thrive. Each possibility represents a separate embodiment of the present invention.

In certain embodiments, the allergy is acute in nature. In certain other embodiments, the allergy is chronic in nature. In other embodiments, the subject is afflicted with a condition associated with delayed type hypersensitivity.

Allergens

The allergic response of methods and compositions of the present invention is, in another embodiment, in response to an inhaled antigen. In another embodiment, the allergic response is a pollen allergy. In another embodiment, the allergic response is dust mite allergy. In another embodiment, the allergic response is venom allergy. In another embodiment, the allergic response is insect bite allergy. In another embodiment, the allergic response is an animal dander allergy. In another embodiment, the allergic response is a response to a surgical implant or device. In another embodiment, the allergic response is directed to an herb selected from the group consisting of Passiflora incarnata, Scutellaria laterifolia, Humulus Lupullus, Melissa officinalis or an extract thereof. In another embodiment, the allergic response is directed to an algae e.g. Spirulina platensis. In another embodiment, the allergic response is selected from the group consisting of a pollen allergy, a dust mite allergy, a venom allergy, an insect bite allergy, an animal dander allergy, an allergy to a surgical implant or device, and an allergy to a component of a species selected from Passiflora incarnata, Scutellaria laterifolia, Humulus Lupullus, Melissa officinalis, and Spirulina platensis. In another embodiment, the allergic response is any other type of allergic response known in the art. Each possibility represents a separate embodiment of the invention.

In another embodiment, the allergy is a pediatric allergy. In another embodiment, the allergy is a pediatric allergy. . In another embodiment, the allergy is an infant allergy. In another embodiment, the allergy is an infant food allergy, e.g. an allergy to infant formula. In another embodiment, the allergy is any other type of pediatric allergy or infant allergy known in the art. Each possibility represents a separate embodiment of the present invention.

In another embodiment, methods and compositions of the present invention are utilized in detecting or monitoring an allergy or allergic disorder suspected to be associated with an ingested allergen. The ingested allergen is in this case utilized as the test substance.

The ingested allergen is, in another embodiment, is a food allergen. As provided herein, methods of the present invention are capable of distinguishing food allergies from other causes of adverse food reactions, e.g. lactose intolerance, irritable bowel syndrome, infectious gastroenteritis, etc. In another embodiment, the allergic reaction is a food allergy or immunologic food sensitivity. In another embodiment, the allergic reaction is manifested by malnutrition. In another embodiment, the allergic reaction is manifested by a gastrointestinal symptom. In other embodiments, the food allergy is chronic in nature. In other embodiments, the subject is afflicted with a condition associated with delayed type food hypersensitivity. Each possibility represents a separate embodiment of the present invention.

A non-limiting example of an immunologic food sensitivity is gluten sensitivity, or more severely, the intestinal disease celiac sprue. Celiac sprue (also known as nontropical sprue, gluten enteropathy and celiac disease) results from an immunologic reaction to dietary gluten contained in wheat, barley, rye, and oats, which results in any degree of intestinal histopathology. By current definitions and classic descriptions, the gluten-induced immunologic process causes villous atrophy and inflammation of the small intestine, in turn, resulting in diarrhea and weight loss from malabsorption of fluid, electrolytes, and dietary nutrients. Some patients are asymptomatic or only have signs of nutritional deficiency, while others have significant gastrointestinal symptoms. Other exemplary food sensitivities are e.g. sensitivity to dietary yeast, particularly Saccharomyces cerevisiae (the yeast utilized in baker's and brewer's yeast, as well as to make fermented foods such as sauerkraut and others), and sensitivities to milk or eggs, e.g. sensitivities to lactalbumin and casein.

In another embodiment, methods and compositions of the present invention are used to screen food allergens, i.e. samples containing an epitope derived from an antigen (typically a protein or peptide) that can be encountered by normal dietary consumption. Exemplary food allergens include, but are not limited to, those derived from milk, eggs, peanuts, soy, wheat, tree-nuts, meat, fish, shellfish and fruit. Test samples for children typically include the most common food antigens to which children are allergic and include, for example, the following: cow's milk antigens (e.g. casein or whole milk extracts); soy protein antigens (e.g. whole soy extracts); peanut antigens (e.g. whole peanut extracts); wheat antigens (e.g. whole extracts, or gluten from wheat); egg antigens (e.g. whole egg extracts); tree-nut antigens, and fish and shellfish antigens. Test samples for adults typically include antigens obtained from various vegetables and fruits, shellfish (e.g. mollusk or crustacean extracts), meat (e.g. poultry or beef extracts), grains and nuts.

Other sources of antigens are well known in the art and are commercially available from a number of sources. For example, the RAST fx5 Pediatric Food Screen contains cow's milk, hen's egg white, wheat, soy, peanut and codfish antigens; the fx1 Tree Nut Screen contains brazil nut, hazelnut, almond, coconut and peanut antigens; the fx2 seafood mix contains codfish, shrimp, blue mussel, tuna, and salmon antigens; the fx3 cereal mix screen contains wheat, oat, maize, sesame seed and buckwheat antigens; and the fx7 mix contains tomato, yeast, garlic, onion and celery antigens. It should be noted, that food extracts or other food allergen samples should be suitable for tissue culture assays, e.g. they should be sufficiently free of exogenous pathogens, cytotoxic agents or any other ingredients that are not suitable for culturing cells under incubation conditions as detailed below.

In other embodiments, the allergen is derived from a naturopathic or homeopathic medicament. In one embodiment, the methods of the invention are useful for diagnosing hypersensitivity to a naturopathic medicament. In another embodiment, the methods of the invention are useful for diagnosing hypersensitivity to a homeopathic medicament. In another embodiment, the methods of the invention are useful for diagnosing hypersensitivity to a food supplement. In another embodiment, the medicaments or supplements comprise plant extracts or herbal preparations (e.g. may contain extracts or preparations of one or more plants or algae, and may also contain minerals and microorganism preparations). For example, without limitation, the test samples including the putative allergens may include extracts of one or more herbs e.g. Passiflora incarnata, Scutellaria laterifolia, Humulus Lupullus, Melissa officinalis, or algae e.g. Spirulina platensis.

Other exemplary herbs that are commonly used in herbal medicine include e.g. Ginkgo biloba, Piper methysticum, Hypericum perforatum, Valeriana officinalis, Panax ginseng, Silybum marianum, Glycyrrhiza glabra, Urtica dioica and Hamamelis virginiana.

In another embodiment, the suspected food allergen is contained in a nutritional supplement. Nutritional supplements take a variety of forms, including beverages, foods (e.g. energy bars, snacks, meals, etc.), gels, and powders for preparation of instants drinks and shakes. Nutritional supplements are preferably administered orally but may also be administered via other routes; i.e. intravenously. Each possibility represents a separate embodiment of the present invention.

In another embodiment, the suspected food allergen is contained in a naturopathic medicament. In another embodiment, the suspected food allergen is contained in a homeopathic medicament. In another embodiment, the naturopathic homeopathic medicament comprises a plant extract. In another embodiment, the naturopathic homeopathic medicament comprises herbal preparation. In these embodiments, the supplement or medicament or a fraction or component thereof is utilized as the test substance. Each possibility represents a separate embodiment of the present invention.

Each type of food allergy represents a separate embodiment of the present invention. In another embodiment, the present invention provides a method for diagnosing hypersensitivity to naturopathic medicament or homeopathic medicament, comprising the step of measuring IFN-gamma release or secretion from a T-cell containing sample of the subject in the presence of the naturopathic or homeopathic medicament or a fraction or component thereof, as described herein. In another embodiment, the naturopathic or homeopathic medicament comprises an extract from an herb selected from the group consisting of Passiflora incarnata, Scutellaria laterifolia, Humulus Lupullus, Melissa officinalis. In another embodiment, the herb is any other type of herb known in the art. In another embodiment, the naturopathic or homeopathic medicament comprises an algae or a product derived therefrom. In another embodiment, the species of algae is Spirulina platensis. In another embodiment, the species of algae is any other type of algae known in the art. Each possibility represents a separate embodiment of the present invention.

In another embodiment, the suspected ingested allergen is contained in an orally administered pharmaceutical compound or pharmaceutical composition. In another embodiment, the ingested pharmaceutical compound is selected from the group consisting of antibiotics, sulfa drugs, and barbiturates. In another embodiment, the ingested pharmaceutical compound is selected from the group consisting of antibiotics, protein or peptide drugs, polysaccharide drugs, oxpentifylline, dopamine agonists, reversible inhibitors of acetylcholineesterase, nitroglycerine, isosorbide dinitrate and mononitrate, opioid drugs, prokinetic drugs, nonsteroidal anti-inflamatory drugs, steroid drugs and corticosteroid drugs, contraceptive or steroidal hormones, immunosuppressants, bronchodilators, anti-anginals and anti-hypertensives, anti-spasmodic agents, anti-colitis agents, anti-arrhythmia agents, anti-neoplastic agents, anti-coagulants, and anti-migraine drugs. In these embodiments, the pharmaceutical compound or pharmaceutical composition or a fraction or component of the pharmaceutical composition is utilized as the test substance. Each possibility represents a separate embodiment of the present invention.

In another embodiment, the present invention provides a method for quantification of an allergic response of a subject to a ingested allergen (e.g. a food allergen or pharmaceutical ingredient), comprising the step of measuring antigen-specific IFN-gamma production by a T-cell containing cell population by a method of the present invention, as described herein, whereby the level of antigen-specific IFN-gamma production indicates in an quantitative fashion the severity of the allergic response.

In another embodiment, the allergy is suspected to be associated with an inhaled allergen, which is utilized as the test substance. In another embodiment, the inhaled antigen of methods and compositions of the present invention is an air-borne allergen. In another embodiment, the air-borne allergen is selected from the group consisting of a pollen, house dust mite feces, bird feces, and animal danders. Each possibility represents a separate embodiment of the present invention.

Pollens associated with inhalation-induced allergies are often from an anemophilous plant. In another embodiment, the pollen is a ragweed pollen. In another embodiment, the pollen is from an anemophilous spring blooming plant, e.g. oak (i.e. a tree of the genus Quercus), hickory (Carya; e.g. pecan or Carya illinoinensis), birch (Betula), alder (Alnus), hazel (Corylus), hornbeam (Carpinus), horse chestnut (Aesculus), willow (Salix), poplar (Populus), plane (Platanus), linden/lime (Tilia) and olive (Olea). In another embodiment, the tree is any other type of tree known in the art. In another embodiment, the pollen is from a grass (Family Poaceae). In another embodiment, the pollen is from an early summer grass. In another embodiment, the grass is selected from the group consisting of ryegrass (Lolium sp.) and timothy (Phleum pratense). In another embodiment, the grass is any other type of grass known in the art. In another embodiment, the pollen is from a weed. In another embodiment, the weed is selected from the group consisting of ragweed (Ambrosia), plantain (Plantago), nettle/parietaria (Urticaceae), mugwort (Artemisia), Fat hen (Chenopodium) and sorrel/dock (Rumex). In another embodiment, the weed is any other type of weed known in the art. Each possibility represents a separate embodiment of the present invention.

House dust mites associated with inhalation-induced allergies are typically the European house dust mite (Dermatophagoides pteronyssinus) and the American house dust mite (Dermatophagoides farinae).

Animal danders associated with inhalation-induced allergies are often from dander of dogs (Canis lupus familiaris), cats (Felis catus), birds, horses (Equus caballus), cows (e.g. Bos taurus and Bos indicus), and pigs (Sus).

Each type of inhaled antigen represents a separate embodiment of the present invention.

In another embodiment, the inhaled antigen is an inhaled pharmaceutical compound. In another embodiment, the inhaled pharmaceutical compound is selected from the group consisting of albuterol, albuterol sulfate, atropine sulfate, beclomethasone, dipropionate, bitolterol mesylate, budesonide, cromolyn sodium, desflurane, dexamethasone sodium, dornase alfa, enflurane, epinephrine, ergotamine tartrate, flunisolide, fluticasone propionate, formoterol fumarate halothane, iloprost, recombinant insulin, ipratropium bromide, isoetharine, isoflurane, isoproterenol, levalbuterol, metaproterenol sulfate, methacholine chloride, mometasone furoate, nedocromil sodium, nicotine, nitric oxide, pentamidine isethionate, pentetate calcium trisodium, pentetate zinc trisodium, pirbuterol acetate, ribavirin, salmeterol xinafoate, sevoflurane, tetrahydrocannabinol, tiotropium bromide, tobramycin, triamcinolone acetonide, and zanamivir. In another embodiment, the inhaled pharmaceutical compound is any other inhaled pharmaceutical compound known in the art. Each possibility represents a separate embodiment of the present invention.

In another embodiment, the present invention provides a method for quantification of an allergic response of a subject to an inhaled antigen, comprising the step of measuring antigen-specific IFN-gamma production by a T-cell containing cell population by a method of the present invention, as described herein, whereby the level of antigen-specific IFN-gamma production indicates in an quantitative fashion the severity of the allergic response.

In another embodiment, the allergy is suspected to be associated with an injected allergen, which is utilized as the test substance.

In another embodiment, the injected antigen of methods and compositions of the present invention is an injected pharmaceutical compound. In another embodiment, the injected pharmaceutical compound is selected from the group consisting of anticonvulsants, recombinant proteins (e.g. insulin preparations, particularly from animal sources of insulin), local anesthetics (e.g., Novocain), and iodine (found in many X-ray contrast dyes. In another embodiment, the injected pharmaceutical compounds is any other injected pharmaceutical compound known in the art. Each possibility represents a separate embodiment of the present invention.

In another embodiment, the allergic response is directed to an insect venom allergen. In another embodiment, the insect is selected from the group consisting of honeybees (Apis), bumblebees (Bombus), hornets (Vespa), wasps (Vespidae), yellow jackets (Vespula and Dolichovespula), and fire ants (Solenopsis). Insect venom allergy (e.g. bee sting allergy) symptoms typically begin with a dry cough and may progress to itching and/or swelling of the eye area, sneezing, and wheezing. These symptoms may be warning signs of a life-threatening anaphylaxis. Symptoms include sudden anxiety and weakness, difficulty breathing, tightness in the chest, very low blood pressure, loss of consciousness, and shock. In some embodiments, the methods of the invention provide for predicting pre-disposition of a subject for developing allergen-related (e.g. insect-venom related) anaphylaxis. Each possibility represents a separate embodiment of the present invention.

In another embodiment, the present invention provides a method for quantification of an allergic response of a subject to an injected antigen, comprising the step of measuring antigen-specific IFN-gamma production by a T-cell containing cell population by a method of the present invention, as described herein, whereby the level of antigen-specific IFN-gamma production indicates in an quantitative fashion the severity of the allergic response.

In another embodiment, the allergy diagnosed by methods of the present invention is associated with an allergen selected from the group consisting of an ingested allergen, an inhaled allergen, and an injected antigen. In another embodiment, the allergy is suspected to be associated with an allergen selected from the group consisting of an ingested allergen, an inhaled allergen, and an injected antigen. In another embodiment, the allergy is suspected to be associated with any other type of antigen that is taken internally (as opposed to a topically administered pharmaceutical composition or an antigen that contacts the skin). Each possibility represents a separate embodiment of the present invention.

In general, a wide variety of allergen preparations are available in the art, and many allergens have been molecularly cloned. For example, cloned allergens include Dermatophagoides pteryonyssinus (Der P1); Lol pl-V from rye grass pollen; various insect venoms including venom from jumper ant Myrmecia pilosula, Apis mellifera bee venom phospholipase A2 (PLA2) and antigen 5S, phospholipases from the yellow jacket Vespula maculifrons and white faced hornet Dolichovespula maculata; a large number of pollen proteins including birch pollen, ragweed pollen, Parol (the major allergen of Parietaria oficinalis) and the cross-reactive allergen Parjl (from Parietaria judaica) and other atmospheric pollens including Olea europaea, Artemisia sp., gramineae, Mellitin and Der p I (house dust mite allergens) etc. allergenic fragments and analogs of these allergens may also be used, as known in the art. U.S. Pat. No. 5,593,877 provides a non-limitative example for cloning and expression of vespid venom enzymes, particularly hyaluronidase and phospholipase. U.S. Pat. No. 6,132,734 discloses cloning of mite allergens.

In another embodiment, the allergen of methods and compositions of the present invention is a chemical compound such as, for example, a polysaccharide, a fatty acid moiety, a protein, etc. Antigen preparations may be prepared by any available technique including, for example, isolation from natural sources, in vivo or in vitro expression of recombinant DNA molecules, or other technique known in the art. Each possibility represents a separate embodiment of the present invention.

In some embodiments, the test sample includes attenuated or killed tumor cells, for evaluating a malignancy in the subject. In other embodiments, the test sample includes tumor-associated antigens (or epitope-containing fragments thereof) including, but are not limited to, p53, p21, S100, EGP-40, MAGE-2, MAGE-3, MUC-1, MUC-2, HER-2, TAG-72 and OFP.

Antigens are commercially available from a plurality of sources. For example, samples of commercially available food supplements, herbal extracts or homeopathic medicaments may be used. It should be noted, that such extracts and allergen samples should be suitable for tissue culture assays, e.g. they should be sufficiently free of exogenous pathogens, cytotoxic agents or any other ingredients that are not suitable for culturing cells under incubation conditions as detailed below.

In another embodiment, the present invention provides a method for quantification of an allergic response of a subject to a food allergen, comprising the step of measuring antigen-specific IFN-gamma production by a T-cell containing cell population by a method of the present invention, as described herein, whereby the level of antigen-specific IFN-gamma production indicates in an quantitative fashion the severity of the allergic response.

In another embodiment, the present invention provides a method for quantification of an allergic response of a subject to an inhaled antigen, comprising the step of measuring antigen-specific IFN-gamma production by a T-cell containing cell population by a method of the present invention, as described herein, whereby the level of antigen-specific IFN-gamma production indicates in an quantitative fashion the severity of the allergic response.

In another embodiment, the present invention provides a method for quantification of an allergic response of a subject to an injected antigen, comprising the step of measuring antigen-specific IFN-gamma production by a T-cell containing cell population by a method of the present invention, as described herein, whereby the level of antigen-specific IFN-gamma production indicates in an quantitative fashion the severity of the allergic response.

Surgical Devices and Stents

In one aspect, there is provided an in vitro method for detecting an allergic response associated with a surgical device implanted in a subject, comprising the step of measuring IFN-gamma production in the presence of a component of the surgical device by a T-cell containing cell population by a method of the present invention, as described herein, whereby an increased level of IFN-gamma production compared to a reference standard indicates the presence of an allergy to the test compound and thus to the surgical device.

In another embodiment, the surgical device is an implant. In another embodiment, the surgical device is a surgical staple. In another embodiment, the surgical device is a stent. In another embodiment, the surgical device is selected from the group consisting of an implant, a surgical staple, and a stent. In another embodiment, the surgical device is any other type of surgical device known in the art.

In another embodiment, the surgical device is a suture. In another embodiment, the suture is an absorbable suture. In another embodiment, the suture is a non-absorbable suture. In another embodiment, the suture is a silk suture. In another embodiment, the suture is a gut suture. In another embodiment, the suture is made of any naturally occurring material known in the art. In another embodiment, the suture is made of a material selected from polypropylene, polyester, and nylon. In another embodiment, the suture is made of a synthetic material. In another embodiment, the suture is any other type of suture known in the art. Each possibility represents a separate embodiment of the present invention.

Surgical implants are well known in the art and are described inter alia in U.S. patent applications 2007/0196421 and 2004/0151753, the contents of which are incorporated herein by reference. Each type of implant represents a separate embodiment of the present invention.

Surgical staples are well known in the art and are described inter alia in U.S. patent applications 2004/0028502 and 2006 /0107645, the contents of which are incorporated herein by reference. Each type of surgical staple represents a separate embodiment of the present invention.

Surgical stents are well known in the art and are described inter alia in U.S. patent applications 2008/0107795 and 2008/0103583, the contents of which are incorporated herein by reference. Each type of surgical stent represents a separate embodiment of the present invention.

In another embodiment, the present invention provides a method for monitoring an allergy or allergic reaction to removal of a surgical implant or stent, comprising the steps of measuring antigen-specific release of IFN-γ protein by T-cell containing cell populations obtained from a subject at multiple time points, as described above.

In the methods of the invention, the device is in some embodiments an orthopedic or cardiovascular implant e.g. prostheses and stents. For example, without limitation, the implant is selected from the group consisting of vascular implants and supports (e.g. intravascular stents and extravascular stents), intraluminal devices, small joint replacements, hip prostheses, knee prostheses, spinal prostheses, shoulder prostheses, joint prostheses, fracture fixation devices, external fixation pins, intramedullary nails, screws, plates, rods, and cages, screws, plates, prosthesis anchors, tacks, staples, vertebral disks, bone pins, clips, and osteointegrated orthopedic devices.

In certain embodiments, the device comprises metal and/or metal alloy components, and the allergen is a metal allergen. In another embodiment, the test sample contains particles obtained from (or otherwise equivalent or corresponding to the composition of) the implantable device, thereby providing high biological relevancy of the test results. For example, the implant is comprised of stainless steel (e.g. ASTM F138, containing Fe, Ni, Cr and Mo), cobalt alloys (e.g. ASTM F75, containing Co, Ni, Cr and Mo), titanium alloys (e.g. ASTM F136, containing Ti, Al and V), gold (e.g. gold-plated hybrid stents), cobalt chromium alloys (e.g. ASTM F1058, containing Fe, Co, Ni, Cr and Mo), tantalum or a titanium alloy (e.g. Nitinol, containing Ni and Ti).

The allergen may be added in any suitable form which is accessible to cells in culture. For example, metal salt solutions, metal beads or microparticles or any other form known in the art may be used. Conveniently, a standardized sample of each suspected allergen will be calibrated for use in the assays. The candidate allergen may also be obtained directly from the implant, e.g. mechanically using a sterile instrument, thereby obtaining a test sample with high biological relevancy. In certain embodiments, wherein beads or particles obtained from the stent or device, or containing a chemical and physical composition corresponding to the assayed stent or device are used, the cells are exposed to the antigen in a manner that is considered to be relevant for periprosthetic exposure, and thereby the clinical relevance of the results is increased.

The methods of the invention provide, in some embodiments, increased sensitivity and specificity compared to hitherto known methods. The methods of the invention enable, in some embodiments, reliable diagnosis and prediction of subtle responses, e.g. those to titanium, vanadium, and tantalum. In one embodiment, the metal is other than nickel. In certain embodiments, the metal is selected from the group consisting of titanium, vanadium and tantalum. In another embodiment, the implant is comprised of an alloy such as the titanium alloy ASTM F136 (containing titanium, aluminum and vanadium).

In another embodiment, the device is a drug-eluting stent. The present invention provides for the first time a method for determining allergic responses to drug-eluting stents, wherein a single assay is used for assessing hypersensitivity to multiple allergen-containing components of the stent.

In various embodiments, the drug is selected from an immune response modifier, an anti-proliferative, an anti-mitotic agent, an anti-platelet agent, a platinum coordination complex, a hormone, an anticoagulant, a fibrinolytic agent, an anti-secretory agent, an anti-migratory agent, an immunosuppressive (e.g. sirolimus), an angiogenic agent, an angiotensin receptor blocker, a nitric oxide donor, a cell cycle inhibitor, a corticosteroid, an angiostatic steroid, an anti-parasitic drug, an anti-glaucoma drug, an antibiotic, a differentiation modulator, an antiviral drug, an anticancer drug (e.g. paclitaxel), and an anti-inflammatory drug.

In other embodiments, the allergic response is associated with in-stent restenosis or late stent thrombosis. In another embodiment, the allergic response is associated with implant failure or rejection. Each possibility represents a separate embodiment of the present invention.

In another embodiment, the present invention provides a method for quantification of an allergic response of a subject to a surgical device, comprising the step of measuring antigen-specific IFN-gamma production by a T-cell containing cell population by a method of the present invention, as described herein, whereby the level of antigen-specific IFN-gamma production indicates in an quantitative fashion the severity of the allergic response.

In another embodiment, the present invention provides a method for evaluating the suitability of an implant or surgical device to a subject prior to implanting. Thus, in another aspect, there is provided an in vitro method for predicting an allergic response to an implantable medical device, comprising the step of measuring antigen-specific IFN-gamma production by a T-cell containing cell population by a method of the present invention, as described herein, whereby an increased level of antigen-specific IFN-gamma production relative to an appropriate reference standard indicates incompatibility of the subject with the implant or surgical device.

Latex Allergies

As provided herein, allergen-induced IFN-γ secretion can be used as an accurate and reliable assay for determining and quantifying the extent of latex allergy. Thus, other embodiments of the invention provide for pre-surgical assessment of allergy to latex gloves and implantable devices comprising latex.

In one aspect, there is provided an in vitro method for detecting a latex allergic response, comprising the step of measuring IFN-gamma production in the presence of latex or a component thereof by a T-cell containing cell population by a method of the present invention, as described herein, whereby an increased level of IFN-gamma production compared to a reference standard indicates the presence of an allergy to the test compound and thus to latex.

In another embodiment, the present invention provides a method for quantification of an allergic response of a subject to latex, comprising the step of measuring latex antigen-specific IFN-gamma production by a T-cell containing cell population by a method of the present invention, as described herein, whereby the level of antigen-specific IFN-gamma production indicates in an quantitative fashion the severity of the allergic response.

Cells

For diagnosing, predicting and/or quantifying an allergy according to the invention, a T cell-containing biological specimen (e.g. a blood sample) is obtained from the subject. The specimen contains a population of cells that secrete IFN-γ when activated by a specific antigen, particularly a T-cell containing cell population. In some embodiments, the cell population is a peripheral blood lymphocyte (PBL) population. In another embodiment, the cell population is a peripheral blood mononuclear cell (PBMC) population. In another embodiment, the cell population is selected from the group consisting of PBL, PBMC, and purified T cells. Methods for obtaining cells suitable for methods of the present invention are known in the art. A non-limiting exemplary method for obtaining PBMC from a blood sample is presented in the Examples below. Methods for purification of T-cells are well known in the art and include use of magnetic beads and fluorescence-activated cell sorting (FACS). Kits are available from Westburg (Netherlands) and also include Dynabeads™ from Invitrogen Corporation. FACS is well known in the art and is described inter alia in U.S. Pat. No. 6,977,156, the contents of which are incorporated herein by reference.

In certain embodiments, the cells are preferably obtained from a subject that is not undergoing drug therapy, particularly administration of anti-inflammatory drugs. For example, for patients treated by anti-histamines and/or steroids, the cell-containing specimen is preferably collected several days after discontinuation of the drug treatment, typically 1-3 weeks after treatment discontinuation.

Incubation Conditions

The cells are then incubated in the presence of the putative allergen(s), under conditions enabling release of IFN-γ by T cells in the presence of an antigen recognized by the T cells. “T cells sensitized to an allergen” are cells that have been previously sensitized to or previously activated by the antigen in vivo (i.e. in the subject). The sensitization is associated with a hypersensitivity reaction in the subject.

The incubation is performed in a suitable vessel, e.g. tissue culture dishes or plates or any other vessel as is known in the art for incubation of cells in suspension. Conveniently, multi-compartment plates such as microtiter plates are used. Typically, the incubation is performed at a temperature of between about 35° C. and 39° C., preferably at about 37° C. The incubation time may be, for example, between about 4 and 50 hours, preferably between about 16 and about 48 hours. For example, the incubation may be performed for about 24 hours. Typically, the cells are incubated at 10⁵-10⁷ cells/mL. For example, 0.5×10⁶-3×10⁶ PBMC/mL may be used. Each possibility represents a separate embodiment of the present invention.

The incubation may be performed in the presence of a T-cell activating agent. For example, incubation may be performed in the presence of agents such as mitogens (e.g. phytohemagglutinin (PHA)), antibodies to T cell-surface structures (e.g. antibodies to the CD3 cell-surface molecule or antibodies to the T cell receptor chains), phorbol esters (e.g. phorbol myristate acetate), or a combination of a phorbol ester and a calcium ionophore, such as ionomycin. Each possibility represents a separate embodiment of the present invention.

IFN-γ Detection

In certain other embodiments, the detection of IFN-γ in each vessel or compartment may be performed using an immunoassay such as an enzyme-linked immunosorbant assay (ELISA) testing kit. In such assays, for example, media samples from each vessel are typically incubated in the presence of an immobilized first specific binding agent (e.g. an antibody) capable of specifically binding IFN-γ. Binding of IFN-γ to the first specific binding agent may be measured using any one of a variety of known methods, such as using a labeled second specific binding agent capable of specifically binding IFN-γ (at a different epitope).

Exemplary specific binding agents include e.g. monoclonal antibodies, polyclonal antibodies, and antibody fragments such as recombinant antibody fragments, single-chain antibodies (scFv) and the like. Single-chain antibodies are small recognition units consisting of the variable regions of the immunoglobulin heavy (V_{H}) and light (V_{L}) chains which are connected by a synthetic linker sequence.

Methods of generating monoclonal and polyclonal antibodies are well known in the art. Antibodies may be generated via any one of several known methods, which may employ induction of in vivo production of antibody molecules, screening of immunoglobulin libraries, or generation of monoclonal antibody molecules by continuous cell lines in culture. Antibody fragments may be obtained using methods well known in the art, including, but not limited to by proteolytic hydrolysis of the antibody or by expression in E. coli or mammalian cells (e.g., Chinese hamster ovary (CHO)) cell culture or other protein expression systems) of DNA encoding the fragment. Single-chain Fvs are prepared by constructing a structural gene comprising DNA sequences encoding the heavy chain variable and light chain variable domains connected by an oligonucleotide encoding a peptide linker. The structural gene is inserted into an expression vector, which is subsequently introduced into a host cell such as E. coli. The recombinant host cells synthesize a single polypeptide chain with a linker peptide bridging the two variable domains.

In some embodiments, various conventional tags or labels may be used, such as a radioisotope, an enzyme, a chromophore or a fluorophore. A typical radioisotope is iodine-125 or sulfur-35. Typical enzymes for this purpose include horseradish peroxidase, horseradish galactosidase and alkaline phosphatase.

Alternately, other immunoassays may be used; such techniques are well known to the ordinarily skilled artisan and have been described in many standard immunology manuals and texts.

In some embodiments, the methods of the invention are suitable for automated or semi-automated analysis, and may enable clinical, medium or high-throughput screening of multiple allergens. For example, automated ELISA systems such as Biotest's Quickstep® ELISA Processor, Maxmat Automated microwell ELISA analyzer (Maxmat S.A., France), or DSX™ Four-Plate System (Dynex Technologies) may conveniently be used.

Analysis

“Significant increase” as used herein refers to an increase of IFN-γ protein levels of statistical significance. The increase is assessed relative to a reference standard, e.g. cells incubated under the same conditions with a control sample lacking the allergen, or cells derived from the same subject at a different time point. Conveniently, a positive result according to the methods of the invention may be determined by calculating the increase in IFN-γ release (as a percentage) for each tested allergen according to the following formula:

% interferon gamma (IFN-γ) increase=(IFN-γ release with the allergen−IFN-γ release without the allergen)/(IFN-γ release without the allergen)×100.

Conveniently, a value higher than the mean percentage increase of IFN-γ measured in control subjects +2 SD (threshold level) is considered a positive IFN-γ test result.

In other embodiments, the methods of the invention enable quantification of the immune response of a subject to an allergen, thus providing a method for monitoring the progress of the subject's hypersensitivity reaction to the allergen. According to the invention, the amount (expressed e.g. as mean percentage increase) of IFN-γ secreted in the presence of the allergen may be quantified and calibrated. The values obtained may be compared e.g. to mean percentage increase values measured in previously performed assays for the same allergen using cells obtained from the same subject, or to mean values representative of non-significant, low, moderate or high immune responses of other individuals to the allergen.

In another embodiment, an increase of at least about 30% in detectable IFN-γ over the level detected for cells (e.g. cells obtained from the same subject at the same time point) incubated under the same conditions with a control sample lacking the allergen (or any other allergen), is considered a positive result.

In another embodiment, there is provided a method of quantifying an allergic response of a test subject within a population of subjects to a putative allergen capable of eliciting a hypersensitivity reaction as detailed herein, comprising the steps of: (a) measuring IFN-γ secretion from a T cell-containing sample of a subject in response to an allegen by a method of the present invention; and (b) calculating a mathematical index γ₁ of the response, wherein the mathematical index γ₁=the ratio of IFN-γ secretion of lymphocytes of the test subject in the presence of the putative allergen to the response without the putative allergen; wherein the subject is deemed to be allergic to the putative allergen when a test sample produces a γ₁ value greater than about one standard deviation above the average of the value for the entire population tested.

In another embodiment, the subject is deemed to be allergic to the putative allergen when a test sample produces a γ₁ value greater than about two standard deviation above the average of the value for the entire population tested.

In another embodiment, the subject is deemed to be allergic to the putative allergen when a test sample produces a γ₁ value greater than about 0.3.

In another embodiment, the method further comprises, after step (b), the steps of: (c) measuring IFN-γ secretion in response to the allergen from an additional T cell-containing sample of the subject, wherein the T cell-containing sample has been obtained at a time point subsequent to that of (a); and (d) calculating a mathematical index γ₂ of the response, wherein the mathematical index γ₂=the ratio of IFN-γ secretion of lymphocytes of the test subject in the presence of the putative allergen to the response without the putative allergen; wherein a γ₂ value greater than about one standard deviation above γ₁ indicates progression of the allergic response.

Kits In another aspect, there is provided a kit suitable for carrying out a method of the invention. In one embodiment, the kit comprises (i) a test sample containing a putative allergen associated with an allergic response selected from pollen allergy, dust mite allergy, venom allergy, drug allergy, and animal dander allergy; and (ii) means for detecting the presence of secreted IFN-γ, as detailed herein.

In another embodiment, the present invention provides a kit suitable for detecting a hypersensitivity reaction associated with administration of a naturopathic or homeopathic medicament, the kit comprising a means of measuring IFN-gamma production from a T-cell containing cell population of a subject as described herein.

In another embodiment, the present invention provides a kit suitable for detecting an allergy to a component of a surgical device, the kit comprising a means of measuring IFN-gamma production from a T-cell containing cell population of a subject as described herein.

In another embodiment, the present invention provides a kit for detecting a latex allergy, the kit comprising a means of measuring IFN-gamma production from a T-cell containing cell population of a subject as described herein.

In another embodiment, the present invention provides a kit suitable for detecting an allergy to an ingested antigen, the kit comprising a means of measuring IFN-gamma production from a T-cell containing cell population of a subject as described herein.

In another embodiment, the present invention provides a kit suitable for detecting an allergy to an inhaled antigen, the kit comprising a means of measuring IFN-gamma production from a T-cell containing cell population of a subject as described herein.

In another embodiment, the present invention provides a kit suitable for detecting an allergy to an injected antigen, the kit comprising a means of measuring IFN-gamma production from a T-cell containing cell population of a subject as described herein.

In another embodiment, a kit of the present invention further comprises instructional material describing how to measure IFN-gamma production from the cell population and optionally in additional how to isolate the cell population.

In another embodiment, each well or test sample contains, in various embodiments, a single type of epitope, a mixture of epitopes (e.g. an extract) obtained from a single putative antigen, or a mixture of certain groups of similar allergens for which one can screen cost-effectively. A positive result reliably indicates that the patient has been sensitized to one or more of the constituent allergens in the mixture, and the sample should be further investigated with individual tests to identify the responsible individual allergens. A negative result with the mixed-allergen test reliably excludes any of the component allergens, thereby obviating any further testing for these allergens.

In another embodiment, a kit of the present invention comprises a plurality of test samples, each test sample containing a putative allergen; and (ii) a means for detecting the presence of IFN-γ protein secreted by T cells. In another embodiment, the kit comprises a plurality of wells or compartments, for testing multiple samples from a single subject. In another embodiment, the plurality of wells or compartments are used for simultaneous testing of sample from multiple subjects. Each possibility represents a separate embodiment of the present invention.

In some embodiments, the kit comprises (i) at least one vessel comprising a test sample containing the at least one putative allergen, wherein the vessel is suitable for incubating a cell population, under conditions enabling release of IFN-γ by cells sensitized to an allergen; and (ii) means for detecting the presence of IFN-γ in the vessel. Such vessels include, for example, tissue culture grade wells e.g. a microtiter plate.

Yet other embodiments of the invention are directed to kits suitable for detecting an allergic reaction to an antigen in a subject, for determining the identity of an allergen associated with hypersensitivity in a subject, for detecting antigen-specific T cells in a subject and/or for quantifying the immune response of a subject to a putative allergen, e.g. wherein the subject is suspected of having an adverse immune reaction associated with administration of naturopathic or homeopathic medicaments or dietary supplements.

In some embodiments, the kit comprises (i) an antigen array distributed over a plurality of vessels, each vessel comprising a test sample containing at least one putative allergen as detailed herein, wherein the vessels are suitable for incubating a cell population, under conditions enabling release of IFN-γ by cells sensitized to an allergen; and (ii) means for detecting the presence of IFN-γ in each vessel.

Test samples containing latex allergens can be produced by methods known in the art. In another embodiment, commercially available extracts (e.g. by Bencard Laboratories, Mississauga, Ontario, and by Stallergenes), latex glove extracts (made using a standardized method of soaking glove material in diluent) and hevea leaves extracts are used. In addition, purified protein or peptide antigen may be purified or synthesized using known recombinant or synthetic methods. A non-limitative example for producing purified latex allergens is described in U.S. Pat. No. 6,759,517.

In another embodiment, the test samples including the putative allergens include extracts of one or more herbs e.g. those described herein. In another embodiment, the means for detecting the presence of IFN-γ comprise an immunoassay. In another embodiment, the immunoassay used in the kit is an enzyme-linked immunosorbant assay (ELISA) testing kit.

In another embodiment, the kit further comprises media suitable for incubating the T cells, and/or reagents suitable for the separation from the sample, such as the separation of PBMCs or T cells from the sample, as detailed above.

In another embodiment, the kit further comprises controls, such as positive or negative controls.

In another embodiment, the kit further comprises directions for using the kit and containers to hold the materials of the kit.

In another embodiment, the present invention provides a kit or apparatus for high-throughput allergy testing, the kit comprising:

a. a means of incubating a T cell-containing cell population from a subject in a culture media comprising a test substance, under conditions enabling release of IFN-γ by the T cell-containing cell population; and b. a means of measuring the amount of IFN-γ protein in the culture media. In another embodiment, the kit further comprises an instruction to compare the amount of IFN-γ present to a reference standard. Each possibility represents a separate embodiment of the present invention.

“High-throughput allergy testing,” as used herein, refers to methods for readily screening dozens of samples. In another embodiment, the methods are amenable for screening hundreds of samples. In another embodiment, the methods are amenable for screening thousands of samples. In another embodiment, the samples are multiple samples from the same subject, wherein the vessels contain a multiplicity of test antigens. In another embodiment, the samples are from a variety of subjects. This may be used to screen a variety of subjects for allergy to a single antigen or a multiplicity of antigen. Each possibility represents a separate embodiment of the present invention.

In another embodiment, methods of the present invention exclude use of radioactivity, thus being amenable to inexpensive, high-volume use not requiring special facilities. Each possibility represents a separate embodiment of the present invention.

In another embodiment, the present invention provides use of:

a. a means of incubating a T cell-containing cell population from a subject in a culture media comprising a test substance, under conditions enabling release of IFN-γ by the T cell-containing cell population; and

b. a means of measuring the amount of IFN-γ protein in the culture media, in the preparation of a kit for detecting an allergy or allergic disorder in a subject, wherein the allergy or allergic disorder is manifested by a symptom selected from the group consisting of renal, respiratory, cardiovascular and gastrointestinal symptoms, whereby a significant increase in the amount of IFN-γ protein relative to the reference standard indicates that the subject is allergic to the test substance. In another embodiment, the kit further comprises an instruction to compare the amount of IFN-γ present to a reference standard. Each possibility represents a separate embodiment of the present invention.

The following Examples are presented in order to more fully illustrate some embodiments of the invention. They should, in no way be construed, however, as limiting the broad scope of the invention.

### Experimental Details Sections

### Example 1

### Determining Allergy to Air-Borne Allergens

An in vitro allergen-induced IFN-γ release test is conducted on lymphocytes obtained from a patient suspected of having allergic rhinitis and from control subjects. Peripheral blood mononuclear cells (PBMC) are obtained by Ficoll-Hypaque gradient centrifugation using standard procedures (Livni et al., 1999). The isolated cells are incubated (2×10⁶/mL) for 24 hours (37° C., 5% CO₂) in M-199H medium (Hank's balanced salt solution; Biological Industries, Beit Haemek, Israel), containing 5% fetal calf serum, PHA-P 200 μg/mL (Bactophytohemagglutinin Purified; Difco Laboratories, Detroit, Mich.) and glutamine (2 mmol/L), with or without the test sample. Each well contains a test sample containing an airborne allergen, as detailed below, or a control sample lacking an allergen. The following allergens are examined: ragweed, sagebrush, lamb's-quarters, plantain, pigweed, dock/sorrel, and tumbleweed. In some experiments, serum free medium is utilized.

After incubation, supernatants are collected using centrifugation (2500 rpm for 25 minutes, 4° C.), and kept at −70° C. until used. The human IFN-γ level is determined in the culture supernatant (pg/mL) using a commercial enzyme-linked immunosorbent assay kit (Quantikine; R and D systems, Minneapolis, Minn).

A positive result is determined by calculating the increase in IFN-γ release (as a percentage) for each drug according to the following formula:

% interferon gamma (IFN-γ) increase=(IFN-γ release with the allergen−IFN-γ release without the allergen)/(IFN-γ release without the allergen)×100

wherein a value higher than 30% (threshold level) is considered a low—to moderately positive IFN-γ test result and a value higher than 60% is considered a highly positive reaction.

### Example 2

### Diagnosis of Acute Interstitial Nephritis Induced by a Naturopathic Herbal Preparation

A 63-year-old white male bearing the medical history of hypertension, adult onset diabetes mellitus type 2, chronic renal failure (Cr-1.4 mg/dL), hyper-cholesterolemia, and chronic obstructive pulmonary disorder was seen for a routine follow up in the nephrology and hypertension clinic. The patient complained of excessive fatigue and presented a two-week-old kidney function test, which showed a moderate impairment (Cr-1.8 mg/dL). Physical examination revealed no abnormalities. The following morning, his creatinine measurement was 7.85 mg/dL, clearly indicating acute renal failure. Other than fatigue, the patient denied symptoms or signs of urolithiasis, irritative or obstructive urinary symptoms, fever, abdominal or flank pain, or sickness or fever in the previous weeks. Besides abnormal creatinine and BUN levels, laboratory results revealed high fasting glucose level (228), mild hyperkalemia (5.1 meq/L), mild hyponatremia (133 meq/L). There was no leukocytosis. Examination of arterial blood gases revealed normal acid-base state. Urinary tests were normal and did not reveal cells or casts. Ultrasound combined with Doppler tests of both kidneys did not indicate any cause for the sudden kidney deterioration.

Severe acute interstitial nephritis (AIN) was diagnosed based on needle biopsy. Oral glucocorticoid treatment (prednisone 1 mg/kg) was begun and was effective. Creatinine gradually declined over the next 8 weeks (Cr-3.5 mg/dL); glucose levels also declined. Another course of steroids was initiated, after which the creatinine level further declined, stabilizing around a level of 1.8 mg/dL, close to the previous value of 1.4 mg/dL. While investigated as to the etiology of his newly diagnosed AIN, the patient described the use of an alternative medicine hypnotic or sedative OTC, “Region-Laila.” Region's leaflet lists 4 active ingredients: Passiflora incarnata 250 mg, Scutellaria laterifolia 50 mg, Humulus Lupullus 50 mg and Melissa officinalis 25 mg.

Seeking to confirm the clinical suspicion of Region causing the unexplained AIN, in-vitro allergen-induced interferon-γ (IFN-γ) release by lymphocytes in the presence reaction to Region was measured. The patient's lymphocytes were separated from heparinized venous blood using Ficoll Hypaque gradient centrifugation and were cultured at 2×10⁶/mL for 24 h at 37° C. and 5% CO₂, in test tubes containing the medium containing phytohemagglutinin and Region (directly dissolved in medium without any modification), or with medium+ to hemagglutinin alone (control). Incubation medium used was M-199H medium (Hank's balanced salt solution; Biological Industries, Beit Haemek, Israel), containing 5% fetal calf serum, PHA-P 200 μg/mL (Bactophyto-hemagglutinin Purified; Difco Laboratories, Detroit, Mich.) and glutamine (2 mmol/L). Test tubes were then centrifuged at 2500 rpm for 25 min at 5° C. Supernatants were collected for the detection of IFN-γ release using the ELISA technique (Biosource, Enco Diagnostics, Petach-Tikva, Israel). IFN-γ release was expressed percentage of its increase calculated by an accepted formula. A significant increase in the IFN-γ release was noted with the drug (261 pg/mL) compared to the control (172 pg/mL; see Table 1). Thus, a 51.7% increase was observed (30% was the threshold for statistical significance), implicating Region in the patient's disease.

TABLE 1
IFN-γ secretion induced by Region, a naturopathic medicament
containing herbal extracts

| | | % IFN-γ |
|---|---|---|
| IFN-γ without Region (pg/mL) | IFN-γ with Region (pg/mL) | increase |
| 172 | 261 | 51.7% |

### Example 3

### Diagnosis of a Hypersensitivity Reaction Induced by a Food Supplement Containing Algal Extracts

An 82-year-old healthy woman presented with bullae, partly hemorrhagic, on the trunk and extremities, secreting erosions, and sub-mammary macerations. The blistering disease developed over a 2-year period, during which she reported taking no drugs. She did, however, begin using a food supplement containing the blue-green alga Spirulina platensis 1 year before the onset of eruption.

At admission, the Nikolsky sign was positive. The first biopsy showed a subepidermal bulla with a denuded surface and sparse perivascular lymphocytic infiltrate with scattered eosinophils. The secondary biopsy, taken during admission, demonstrated an intra- and subcorneal vesicular dermatitis with slight superficial acantholysis. Direct immunofluorescence disclosed IgG and C3 at the dermatoepidermal junction. Indirect immunofluorescence was also positive at the dermatoepidermal junction. A salt split test demonstrated IgG, IgM and C3 on the upper side of the bulla, immunoblotting of the serum was negative and showed no pemphigoid or pemphigus antigens. Enzyme-linked immunosorbent assay (ELISA) for desmoglein 1 and 3 antibodies was also negative.

Diagnostic investigations for neoplasia, Wood's lamp exposure of urine, purified protein derivative (PPD) and antinuclear antibody were negative.

With a diagnosis of mixed immunoblistering disorder exhibiting features of bullous pemphigoid and pemphigus foliaceus, 60 mg prednisone was begun and gradually tapered, and topical treatment was begun with creams containing silver sulfadiazine and triamcinolone/neomycin. Suspecting the involvement of Spirulina in the disease, the food supplement was stopped. With this treatment, the patient's condition steadily improved, and no appearance of new blisters was observed.

An in vitro IFN-γ release test with the Spirulina-containing food supplement was performed. A 19% increase in IFN-γ secretion was observed compared to non-activated cells, i.e. cells obtained from the subject that were incubated without the allergen (% of control). Clearly, a much higher increase in IFN-γ secretion would have been obtained were the patient not treated with 20 mg prednisone at the time. Thus, the test is considered positive.

Three months after completion of the prednisone treatment, and with avoidance of the Spirulina-containing food supplement, the patient was free of lesions with no reoccurrence.

### Example 4

### In Vitro Testing for Compatibility of a Coronary Stent

An in vitro allergen-induced IFN-γ release test is conducted on lymphocytes obtained from a patient, prior to implantation of a coronary stent, and from control subjects. Peripheral blood mononuclear cells (PBMC) are obtained by Ficoll Hypaque gradient centrifugation using standard procedures (Livni et al., 1999). Typically, the isolated cells are incubated (2×10⁶/mL) for 24 hours (37° C., 5% CO₂) in M-199H medium (Hank's balanced salt solution; Biological Industries, Beit Haemek, Israel), containing 5% fetal calf serum, PHA-P 200 μg/mL (Bactophytohemagglutinin Purified; Difco Laboratories, Detroit, Mich.) and glutamine (2 mmol/L), with or without the test sample. Each well contains a test sample obtained from the implant, as detailed below, or a control sample lacking an allergen.

A sample of the stent material, containing bioequivalent allergen-containing particles, is obtained by scraping each stent with a sterile blade. The following stents are examined: Nitinol, Cordis Palmaz-Schatz and Crossflex stents.

After incubation the supernatants are collected using centrifugation (2500 rpm for 25 minutes, 4° C.), and kept at −70° C. until used. The human IFN-γ level is determined in the culture supernatant (pg/mL) using a commercial enzyme-linked immunosorbent assay kit (Quantikine; R and D systems, Minneapolis, Minn.).

A positive result is determined by calculating the increase in IFN-γ release (as a percentage) for each drug according to the following formula:

% interferon gamma (IFN-γ) increase=(IFN-γ release with the allergen−IFN-γ release without the allergen)/(IFN-γ release without the allergen)×100

wherein a value higher than 30% (threshold level) is considered a positive IFN-γ test result.

### Example 5

### In Vitro Testing for Latex Allergy

An in vitro allergen-induced IFN-γ release test was conducted as described in Example 1, on PBMC obtained from a subject with a history of latex allergy. The test samples contained a latex sample.

The results are presented in Table 2 as percentage of detected IFN-γ compared to non-activated cells, i.e. cells obtained from the subject that were incubated without the allergen (% of control).

TABLE 2
hypersensitivity to latex

| Assay | Result |
|---|---|
| IFN-γ release (% of control) | 177 |
| Skin test - wheal size (mm) | 15 × 8 |

As can be seen in Table 2, the subject tested positive to latex (i.e. a significant reaction was determined for the allergen). These results are in correlation with results obtained previously with a scratch skin test performed using standard procedure.

### Example 6

### Determination of the Cause of an Implant-Associated Allergic Response

An in vitro allergen-induced IFN-γ release test was conducted on lymphocytes obtained from a patient suspected of having an implant-associated allergic response. The subject was implanted with a hip prosthesis comprised of an alloy containing titanium and vanadium (Zimmer). PBMC were obtained, and the assay was performed as described in Example 1. Test samples contained titanium or vanadium allergens obtained commercially: vanadium powder (cat. No. 26,293-5) and titanium (IV) oxide (No. T8141) were obtained from Sigma-Aldrich Chemie Gmbh, Germany, and diluted 1:5 (w/v) in sulfuric acid, then 1:10,000 in culture media.

The results are presented in Table 3 as percentage of detected IFN-γ compared to non activated cells, i.e. cells obtained from the subject that were incubated without the allergen (% of control).

TABLE 3
hypersensitivity to titanium or vanadium

| Allergen | IFN-γ release (% of control) |
|---|---|
| Titanium | 157 |
| Vanadium | 98 |

As can be seen in Table 3, the subject tested positive to (i.e. a significant reaction was determined for) titanium, but not to vanadium. Thus, the implant-related hypersensitivity in the subject is associated with the titanium components of the implant. From analysis of the results, a replacement implant may contain vanadium and alloys thereof, but should not contain titanium.

### REFERENCES

Asero R, Ballmer-Weber B K, Beyer K, Conti A, Dubakiene R, Fernandez-Rivas M, Hoffmann-Sommergruber K, Lidholm J, Mustakov T, Oude Elberink J N, Pumphrey R S, Stahl Skov P, van Ree R, Vlieg-Boerstra B J, Hiller R, Hourihane J O, Kowalski M, Papadopoulos N G, Wal J M, Mills E N, Vieths S. IgE-mediated food allergy diagnosis: Current status and new perspectives. Mol Nutr Food Res. 2007 January; 51(1):135-47

Colson C R, De Broe M E. Kidney injury from alternative medicines. Adv Chronic Kidney Dis. 2005 July; 12(3):261-7

Goldberg I, Gilburd B, Kravitz M S, Kivity S, Chaim B B, Klein T, Schiffenbauer Y, Trubniykovr E, Brenner S, Shoenfeld Y. A novel system to diagnose cutaneous adverse drug reactions employing the cellscan—comparison with histamine releasing test and INF-gamma Releasing Test. Clin Dev Immunol. 2005 March; 12(1):85-90.

Goldberg I, Gilburd B, Shovman O, Brenner S. Clinical and laboratory assays in the diagnosis of cutaneous adverse drug reactions. Isr Med Assoc J. 2004 January; 6(1):50-1.

Halevy S, Cohen A, Livni E. Acute generalized exanthematous pustulosis associated with polysensitivity to paracetamol and bromhexine: the diagnostic role of in vitro interferon-gamma release test. Clin Exp Dermatol. 2000 November; 25(8):652-4.

Halevy S, Cohen A D, Grossman N. Clinical implications of in vitro drug-induced interferon gamma release from peripheral blood lymphocytes in cutaneous adverse drug reactions. J Am Acad Dermatol. 2005 February; 52(2):254-61.

Halevy S, Cohen A D, Grossman N. In vitro interferon-gamma release—a laboratory diagnosis of cutaneous adverse drug reactions. Harefuah. 2001 February; 140(2):121-4, 190.

Halevy S, Cohen A D, Livni E. The diagnostic role of the in vitro interferon-gamma release test in Stevens-Johnson syndrome. Int J Dermatol 1999; 38:835-40.

Halevy S, Cohen A D, Lunenfeld E, Grossman N. Autoimmune progesterone dermatitis manifested as erythema annulare centrifugum: Confirmation of progesterone sensitivity by in vitro interferon-gamma release. J Am Acad Dermatol. 2002 August; 47(2):311-3.

Halevy S, Gold I, Cohen A D, Grossman N. In vitro interferon-gamma release test in the diagnosis of drug-induced erythema nodosum. Isr Med Assoc J. 2004 January; 6(1):59-60.

Halevy S. In-vitro tests for adverse drug reactions based on cytokine release. In: Kauppinen K, Alanko K, Hannuksela M, Maibach H, editors. Skin reactions to drugs. Boca Raton (Fla.). CRC Press LLC; 1998. p. 119-31.

Hallab N J, Mikecz K, Vermes C, Skipor A, Jacobs J J. Differential lymphocyte reactivity to serum-derived metal-protein complexes produced from cobalt-based and titanium-based implant alloy degradation. J Biomed Mater Res. 2001 Sep. 5; 56(3):427-36

Koga T, Imayama S, Hori Y. In vitro release of interferon-gamma by peripheral blood mononuclear cells of a patient with carbamazepine-induced allergic drug eruption in response to stimulation with carbamazepine. Contact Dermatitis. 1995 March; 32(3):181-2.

Kubota Y, Koga T, Nakayama J. In vitro released interferon-gamma in the diagnosis of drug-induced anaphylaxis. Eur J Dermatol 1999; 9:559-60.

Livni E, Lapidoth M, Halevy S. In vitro release of interferon-gamma and macrophage migration inhibition factor in drug-induced urticaria and angioedema. Acta Derm Venereol. 1999 January; 79(1):18-22.

Markowitz G S, Perazella M A: Drug-induced renal failure: A focus on tubulointerstitial disease. Clin Chim Acta 351: 31-47, 2005

Spanou Z, Keller M, Britschgi M, Yawalkar N, Fehr T, Neuweiler J, Gugger M, Mohaupt M, Pichler W J. Involvement of drug-specific T cells in acute drug-induced interstitial nephritis. J Am Soc Nephrol 2006; 17(10):2919-27.

Thomas P, Bandl W D, Maier S, Summer B, Przybilla B. Hypersensitivity to titanium osteosynthesis with impaired fracture healing, eczema, and T-cell hyperresponsiveness in vitro: case report and review of the literature. Contact Dermatitis 2006; 55(4):199-202.

Wohl Y, Goldberg I, Sharazi I, Brenner S. A case of paracetamol-induced acute generalized exanthematous pustulosis in a pregnant woman localized in the neck region. Skinmed. 2004 January-February; 3(1):47-9.

Wohl Y, Goldberg I, Shirazi I, Brenner S. Chlorpyrifos exacerbating pemphigus vulgaris: a preliminary report and suggested in vitro immunologic evaluation model. Skinmed. 2006 May-June; 5(3): 111-3.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A method for detecting an allergy or allergic disorder in a subject, said method comprising the steps of:
a) incubating a T cell-containing cell population from said subject in a culture media comprising a test substance, under conditions enabling release of IFN-γ by said T cell-containing cell population;
b) measuring the amount of IFN-γ protein in said culture media; and
c) comparing said amount of IFN-γ protein to a reference standard,
wherein said allergy or allergic disorder is manifested by a symptom selected from the group consisting of renal, respiratory, cardiovascular and gastrointestinal symptoms and is not a cutaneous adverse drug reaction, whereby a significant increase in said amount of IFN-γ protein relative to said reference standard indicates that said subject is allergic to said test substance.

2. The method according to claim 1, wherein said allergy or allergic disorder is selected from the group consisting of: food allergy, an allergy suspected to be associated with the presence of said test substance in a surgically implanted device, and an allergy suspected to be associated with a nutritional supplement or a naturopathic or homeopathic medicament, a pollen, a dust mite, an animal dander, and insect venom.

3. The method according to claim 1, further comprising the steps of:
d) incubating a second T cell-containing cell population from said subject in a second culture media, wherein said second culture media comprises said test substance and is identical to the culture media utilized in step (a) of claim 1, under the conditions utilized in step (a) of claim 1, wherein said second T cell-containing cell population has been obtained from said subject at a time point subsequent to performing the method of claim 1;
e) measuring the amount of IFN-γ protein in said second culture media; and
f) comparing said amount of IFN-γ protein in said second culture media to the amount of IFN-γ protein in the culture media of claim 1,
whereby a statistically significant alteration in said amount of IFN-γ protein in said second culture media relative to the amount of IFN-γ protein in the culture media of claim 1 is indicative of alteration in the magnitude of said allergy or allergic disorder.

4. A method for determining a predisposition of a subject to developing an allergy or allergic disorder to a test substance, said method comprising the steps of:
a) incubating a T cell-containing cell population from said subject in a culture media comprising said test substance, under conditions enabling release of IFN-γ by said T cell-containing cell population;
b) measuring the amount of IFN-γ protein in said culture media; and
c) comparing said amount of IFN-γ protein to a reference standard,
wherein said allergy or allergic disorder is manifested by a symptom selected from the group consisting of renal, respiratory, cardiovascular and gastrointestinal symptoms and is not a cutaneous adverse drug reaction, whereby a significant increase in said amount of IFN-γ protein relative to said reference standard indicates that said subject is allergic to said test substance.

5. The method according to claim 1, wherein said reference standard is obtained by incubating under said conditions an additional aliquot of said T cell-containing cell population in an additional aliquot of the culture media, wherein said additional aliquot of the culture media lacks said test substance but otherwise is identical to the culture media of claim 1, or wherein said reference standard is obtained by performing steps (a) and (b) on a T cell-containing cell population from a control subject.

6. The method according to claim 1, wherein an increase of at least 30% in said amount of IFN-γ protein over said reference standard is considered a positive result.

7. The method according to claim 1, wherein said allergy or allergic disorder is suspected to be associated with ingestion of said test substance and wherein said allergy is food allergy.

8. The method of claim 7, wherein the food allergy is associated with at least one of a milk allergen, an egg allergen, a peanut allergen, a soy allergen, a wheat allergen, a tree-nut allergen, a meat allergen, a fish allergen, a shellfish allergen and a fruit allergen.

9. The method of claim 1, wherein the allergy is chronic in nature.

10. The method of claim 7, wherein the subject is afflicted with a condition associated with delayed type food hypersensitivity.

11. The method according to claim 1, wherein said allergic disorder is selected from the group consisting of eosinophilic gastroenteropathy, a dietary protein gastroenteropathy, and celiac disease.

12. The method according to claim 1, wherein said test substance is contained in a nutritional supplement or a naturopathic or homeopathic medicament containing said test substance.

13. The method of claim 1, wherein the subject is afflicted with interstitial nephritis.

14. The method according to claim 1, wherein said allergy or allergic disorder is suspected to be associated with the presence of said test substance in a surgically implanted device.

15. The method of claim 14, wherein the device is an orthopedic or cardiovascular implant.

16. The method of claim 14, wherein the allergen is a metal allergen other than nickel.

17. The method of claim 16, the metal is selected from the group consisting of titanium, vanadium and tantalum.

18. The method of claim 14, wherein the test sample contains particles corresponding to the composition of the implantable device.

19. The method of claim 14, wherein the device is a drug eluting stent.

20. The method of claim 14, wherein the allergic response is associated with in-stent restenosis or late stent thrombosis.

21. The method of claim 14, wherein the allergic response is associated with implant failure or rejection.

22. The method according to claim 1, wherein the step of measuring the amount of IFN-γ protein is performed using an immunoassay.

23. The method of claim 1, wherein: step a) comprises applying the cells to a plurality of compartments, each compartment comprising a test sample containing at least one putative allergen suspected of being associated with the allergic response in said subject, and incubating said cells in each compartment, under conditions enabling release of IFN-γ by T cells sensitized to the at least one allergen; and step b) comprises detecting the presence of IFN-γ in each compartment.

24. A kit suitable for detecting a hypersensitivity reaction, wherein said kit comprises (i) a plurality of test samples, each test sample comprising at least one putative allergen; and (ii) a means for detecting the presence of IFN-γ protein secreted by T cells, wherein the hypersensitivity reaction is associated with an allergy selected from the group consisting of: food allergy, an allergy suspected to be associated with the presence of said allergen in a surgically implanted device, and an allergy suspected to be associated with a nutritional supplement or a naturopathic or homeopathic medicament, a pollen, a dust mite, an animal dander, and insect venom.

25. The kit of claim 24, wherein the means for detecting the presence of IFN-γ is an immunoassay.

26. The kit of claim 24, wherein the test samples comprise at least one herbal extract selected from the group consisting of extracts of Passiflora incarnata, Scutellaria laterifolia, Humulus Lupullus, Melissa officinalis and Spirulina platensis.

27. The kit of claim 24, wherein the test samples comprise at leas one putative food allergen selected from the group consisting of a milk allergen, an egg allergen, a peanut allergen, a soy allergen, a wheat allergen, a tree-nut allergen, a meat allergen, a fish allergen, a shellfish allergen and a fruit allergen.

28. The kit of claim 24, wherein the test samples comprise at least one putative allergen selected from the group consisting of titanium and vanadium allergens, or wherein the test samples contain particles corresponding to the composition of an implantable device.

29. A kit for high-throughput allergy testing according to claim 24, said kit comprising: (i) an antigen array distributed over a plurality of vessels, each vessel comprising a test sample containing the at least one putative allergen, wherein the vessels are suitable for incubating a cell population, under conditions enabling release of IFN-γ by cells sensitized to an allergen; and (ii) means for detecting the presence of IFN-γ in each vessel.
